(19) United States
(12) Patent Application Publication
(10) Pub. No.: US 2016/0130525 A1
(43) Pub. Date: May 12, 2016

(54) CONTINUOUS DRY MILLING METHOD OF WHOLE GRAIN COMPONENT EXTRACTION

(71) Applicant: Donald L. FISK, Rolla, MO (US)

(72) Inventor: Donald L. FISK, Rolla, MO (US)

(21) Appl. No.: 14/897,395

(22) PCT Filed: Jun. 9, 2014
(86) PCT No.: PCT/US14/41543

§ 371 Date: Dec. 10, 2015

Related U.S. Application Data
(60) Provisional application No. 61/833,182, filed on Jun. 10, 2013.

(51) Int. Cl. C11B 1/10; C07K 14/415; B02C 9/04

(52) U.S. Cl. CPC C11B 1/10; B02C 9/04; C07K 14/415; A23L 1/1025; A23L 1/0152; A23V 2002/00

(57) ABSTRACT

Oil and protein are extracted from a plant-based feedstock using an extruder and a static mixer along with absolute ethanol and supercritical CO₂. The overall process is a dry milling process which preserves the quality of the oil and the protein component of the plant-based feedstock.

### BACKGROUND OF THE INVENTION

1. Field Of Invention

This Invention relates to a method for extracting oil and protein from a plant-based products and more particularly for extracting corn oil and corn protein from corn kernels.

2. Prior Art

Corn kernels are made up of primarily four components, namely, starch, oil, protein, and the hull. The hull is primarily a cellulosic or hemi cellulosic material. There are two main processes for separating the components of a corn kernel, one is a wet milling process and the other is a dry milling process. In both processes, there is a need for maximizing the efficiency of extraction so as to enhance the amount of each of the components extracted after the kernel has been fractionated into its separate components. There is also a need for efficient operation for separating out the various components and recovering the same.

### SUMMARY OF THE INVENTION

Applicant has discovered a process for fractionating and recovering the various components of a plant-based product such as a corn kernel. The process uses a conventional extruder coupled with a static mixer to fractionate the plant-based product in conjunction with the addition of a solvent and supercritical carbon dioxide during the fractionating process. It has been found that such a process greatly enhances the oil and protein extraction from the plant-based product. Such a process is deemed to be a dry milling process since water is not introduced during the process and thus does not fall into the category of wet milling.

Subjecting the plant based product to an extended period of time in a supercritical state is an important aspect of the invention. This extended period of time in a supercritical state is made possible by the use of the static mixer. The static mixer has a die at its exit end such that the plant based product is maintained in a supercritical state throughout its residence in the static mixer. This extended period of time at a supercritical state is an important element in obtaining good fractionation of the various components of the plant based product.

The process of the present Invention can be summarized by the following items:

1. A method for extraction of oil and protein from a plant based product the method comprising:

forming a feedstock from a plant based product having an oil component, a protein component, a starch component and a cellulose component, and a moisture content of to about 15.5 to about 16.5% by weight;

subjecting the feedstock to pressure and shear to cause the feedstock to fractionate into an oil component, a protein component, a starch component and a cellulosic component;

holding the fractionated feedstock in a vessel with agitation for a period of time to form a solid phase having the starch component and the cellulosic component, and a liquid phase having the oil component and the protein component;

separating the solid phase from the liquid phase;

separating the liquid phase into the oil component and protein component;

collecting the oil component separate from the protein component; and

collecting the protein component separate from the oil component.

2. The method of item 1 wherein the plant based product is a product from maize, sorghum, or wheat.

3. The method of item 2 wherein the product is corn kernels, wheat kernels or sorghum grain.

4. The method of item 1, wherein forming the feedstock includes determining the moisture content of the feedstock; and adjusting the moisture content of the feedstock to about 15.5% to about 16.5% by wt., if the determined moisture content is outside a range of about 15.5% to about 16.5% by wt.

5. The method of item 1, wherein forming the feedstock includes milling the feedstock to a size of about 20 mesh to about 30 mesh.

6. The method of item 5 wherein the milling is conducted by a hammer mill, a burr mill or a roller mill.

7. The method of item 1, wherein subjecting the feedstock to pressure and shear includes adding a solvent to the feedstock.

8. The method of item 1, wherein subjecting the feedstock to pressure and shear includes adding a supercritical fluid to the feedstock.

9. The method of item 7, wherein the solvent is added in an amount of about 5 to 50% by weight based on the weight of dry feedstock.

10. The method of item 8, wherein the supercritical fluid is added in an amount of about 10% to about 90% by weight based on the dry weight of feedstock.

11. The method of item 1, wherein the subjecting the feedstock to pressure and shear is conducted at a temperature of about 50° F. to about 180° F., a pressure of about 1,000 psi to about 5,000 psi, and a period of time of about 0.5 to about 5 minutes.

12. The method of item 11, wherein subjecting the feedstock to pressure and shear includes feeding the feedstock to an extruder which pumps the feedstock through a static mixer.

13. The method of item 12, wherein the extruder is a twin screw extruder operated at about 5 to 1500 rpm.

14. The method of item 11, wherein the static mixer has an outlet with a die to restrict outflow of the fractionated feedstock.

15. The method of item 1, wherein holding the fractioned feedstock in the vessel is conducted at a temperature of about 80° F. to about 120° F., atmospheric to below atmospheric pressure, and a period of time of about 0.5 hours to about 1.5 hours.

16. The method of item 1, wherein the separated solid phase is dried to remove the solvent.

17. The method of item 16, wherein the starch component is separated from the cellulose component.

18. The method of item 16, wherein the drier is a Wyssmont Type Drier.

19. The method of item 7, wherein the solvent is absolute ethanol.

20. The method of item 8, wherein the supercritical fluid is supercritical carbon dioxide.

### BRIEF DESCRIPTION OF THE DRAWINGS

FIG. 1 is an overview of the process of the present Invention;

FIG. 2 is a more detailed overview of the process; and

FIG. 3 is a more detailed flow diagram of the present process.

### DETAILED DESCRIPTION OF THE INVENTION

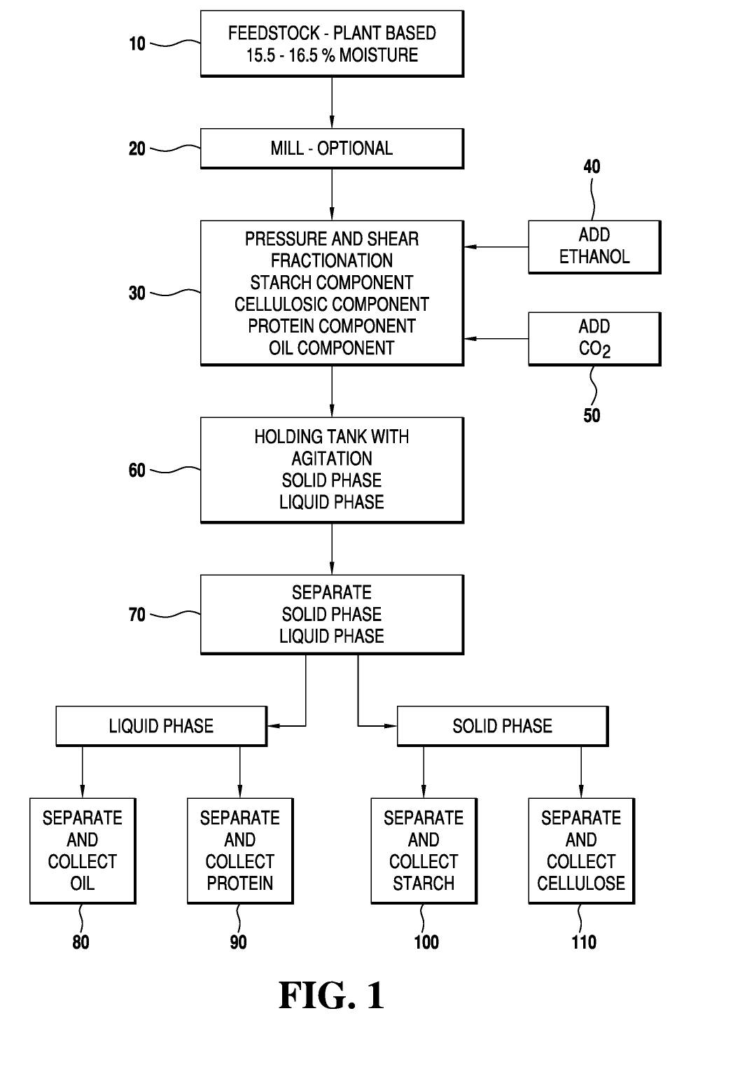

FIG. 1 illustrates the basic steps in the process as shown in FIG. 1. The feedstock 10 plant-based material having a moisture content of about 15.5 to about 16.5% is obtained.

Next, this feedstock can optionally be milled 20 to about 20 mesh to about 30 mesh to reduce the size.

The feedstock, milled or unmilled, is then subject to a pressure and shear step 30 wherein the plant based feedstock is fractionated into a starch component, a cellulosic component, a protein component and an oil component. During the fractionation, solvent 40, preferably ethanol, can be added and supercritical fluid 50, preferably carbon dioxide, can be added.

After the shear and pressure step, the fractionated feedstock is then fed to holding tank 60 in which it is agitated and a solid phase and liquid phase are formed. The solid phase contains the starch component which is destructurized and the cellulosic component while the liquid phase contains the protein component and the oil component of the fractionated feedstock.

Next the fractionated feedstock which is in holding tank 60 is separated into the solid phase and the liquid phase as shown in 70.

Subsequently, the liquid phase is separated into the oil component 80 and the protein component 90 while the solid phase is separated into the starch component 100 and the cellulose component 110.

The plant-based product used for the feedstock is suitably from corn or (maize), sorghum or wheat. The product itself is either a corn kernel, a wheat kernel or a sorghum grain. As is generally understood, each one of these products has an exterior hull which is made up primarily of a cellulosic or hemi cellulosic material while the interior contains the starch, the protein and the oil.

An excellent secondary feedstock is the hominy by-product of the dry milling process because it contains high levels of oil, protein, and cellulose and is already finely ground to a desired size.

In operating the present Invention, the feedstock must have the moisture content of about 15% to about 16.5% by wt. If, for any reason, the moisture content of the feedstock is not within this range, then the moisture content can be adjusted by either adding water or by conducting a drying process. Both adding water and a drying process is done in a conventional manner using conventional equipment. The moisture content is calculated using conventional techniques.

After obtaining the feedstock and adjusting its moisture content as necessary, the feedstock can be subjected to a milling process so as to reduce the size of the feedstock. Suitably, the feedstock is reduced to be able to pass through a mesh in the range of about 20 mesh to about 30 mesh. A mesh of 20 corresponds to a particle size of about 0.8 millimeters and 30 mesh corresponds to a particle size of about 0.5 millimeters. The size of the feedstock can be milled in a conventional manner using conventional equipment, such as a hammer mill, a burr mill and a roller mill. The roller mill is preferred as it will minimize the amount of fines which are produced. Fines generally are produced from the starch component and thus form a flour. Having a flour component is non beneficial.

The next step of the process involves subjecting the feedstock to pressure and shear so as to cause the feedstock to fractionate into its four main elements, namely, an oil component, a protein component, a starch component and a cellulosic component. The cellulosic component generally comprises the hull which is made up of cellulose and hemi cellulose.

During the pressure and the shearing step, a mid polarity or a polar solvent is added. Such a solvent helps to extract the oil components from the other parts of the feedstock. Additionally, a supercritical fluid can be also added during the pressure and shearing step in order to enhance the extraction of the oil from the feedstock.

Suitable solvent for use in the present Invention is ethanol and specifically absolute ethanol which is an ethanol solution comprising 90% to 100% ethanol with the remainder of water. Preferably, a 100% of the water has been removed from the solution.

The supercritical fluid is preferably supercritical carbon dioxide. Supercritical carbon dioxide is carbon dioxide which has a minimum of 1100 psi at a temperature of 88° F. At this temperature and pressure, supercritical carbon dioxide is a fluid having both gas and liquid properties. Specifically, it fills a container like a gas but has the density more of that of a liquid. Supercritical CO₂ is a conventional product well known to those of skill in the art.

The amount of absolute ethanol used in the present Invention is suitably about 5 to about 50% by weight based on the dry weight of the feedstock. More preferably, the amount of absolute ethanol is about 20 to about 30% by weight of the dry feedstock.

The supercritical fluid is suitably used in an amount of about 10 to about 90% by weight based on the dry weight of the feedstock and, more preferably, about 25 to about 50% by weight of the feedstock. Even more preferred is about 10 to about 25% by weight of the dried feedstock.

Suitably, the supercritical carbon dioxide has a temperature in the range of about 80° F. to about 400° F. and, more preferably, a temperature of about 80° F. to about 300° F. The pressure of the supercritical CO₂ is suitably about 1100 psi to about 10000 psi and, more preferably, about 1100 psi to about 5000 psi.

Preferably, the solvent is added to the feedstock during the pressure and shear step before the addition of the supercritical fluid.

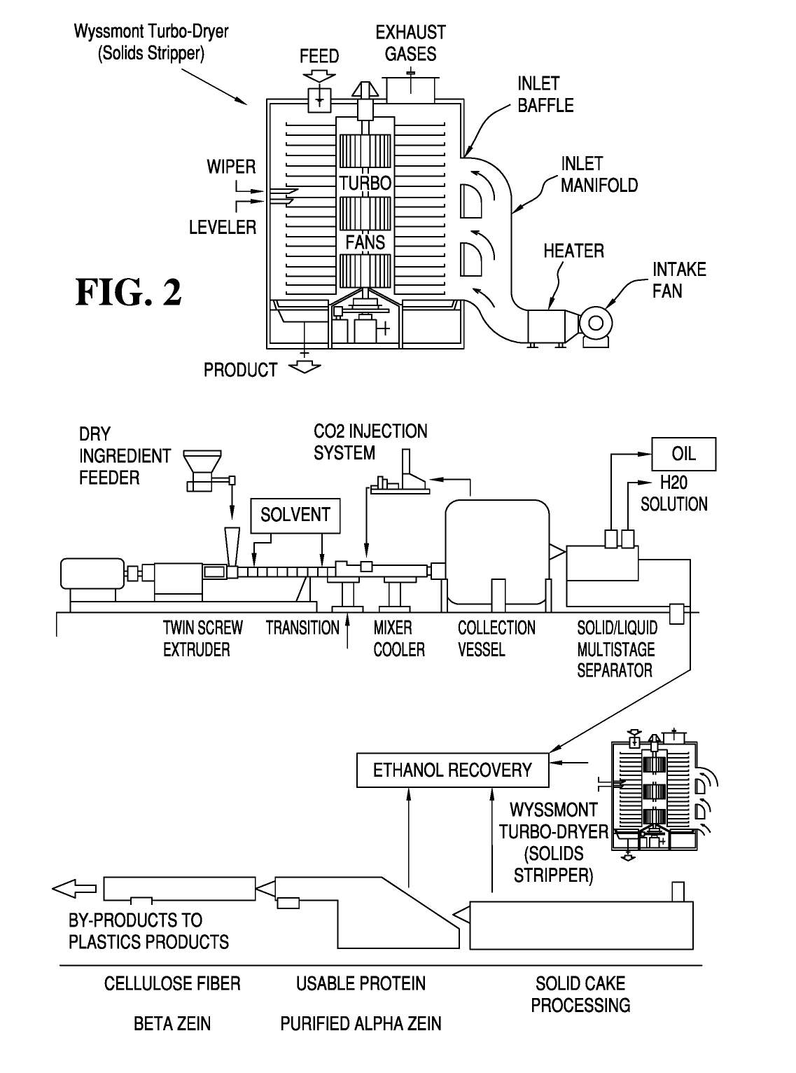

The step of subjecting the feedstock to pressure and shear is suitably accomplished by using an extruder coupled to a static mixer. Such a coupling is shown in FIG. 2 of the Application.

Suitable extruders used in the present Invention include twin screw extruders. The extruder provides the pumping action to move the feedstock through the static mixer. The extruder also provides pressure and shear to the feedstock. If the feedstock has not been subject to the optional milling step, the twin screw extruder will also perform a step of milling the feedstock.

Twin screw extruder is operating in a conventional manner. Suitably, the twin screw extruder is operating at a speed of about 5 to about 1500 rpm's, more suitably, about 400 to about 1000 rpm and even more suitably, about 600 to about 800 rpm's. The temperature during extrusion, while in the extruder, is suitably about 60 to about 120° F. and, more suitably, about 80 to about 100° F.

The static mixer is a conventional piece of equipment used in a conventional manner. A suitable static mixer is ProMix Type SMB Plus 50 with cooling jacket. A typical static mixer is a housing with internal elements or baffles which cause the material to mix and blend and provide shear to the fractionated feedstock as it moves through the static mixer. Typically, these internal elements have helical shapes or plate shapes and are respectively referred to as helical-type static mixers and plate-type static mixers.

Suitably, the length of the static mixer in proportion to the internal diameter of the extruder barrel given by the equation 7 X=L wherein X is the extruder internal diameter and L=the internal length of the mixer.

There is a die positioned at the end of the static mixer so as to maintain the pressure on the feedstock as it moves through the combined extruder and mixer. The opening of the die is arranged so as to the control the exit speed of the feedstock from the static mixer.

Suitably, the solvent is injected into the extruder in the first 30% of the length of the extruder and the last 30% of the length of the extruder, the beginning of the extruder being where the feedstock is introduced and the end of the extruder being the point where it is coupled to the static mixer.

The supercritical fluid is suitably injected at the entrance to the static mixer. This is the point where the static mixer is coupled to the outlet of the extruder. However, it is understood that the supercritical fluid can be injected at various points along the extruder barrel and the mixer.

Due to the extruder creating pressure and shear plus the solubilizing effects of the supercritical fluid, the static mixer performs two tasks. First, it causes the feedstock to be thoroughly saturated and intermittently mixed with the solvent and fluid. The second effect is the pressure drop which occurs upon the exiting of the feedstock from the mixer. The exit of the feedstock from the mixer causes the starch to be destructurized as the solvent and fluid rapidly disperse from the feedstock.

The temperature of the feedstock as it passes through the extruder and the static mixer is about 50° F. to about 180° F. and, more suitably, about 70 to about 145° F.

The pressure inside the extruder and the static mixer while the feedstock is moving therethrough is about 1000 psi to about 5000 psi and, more suitably, about 1500 psi to about 2250 psi.

The residence time that the feedstock remains in the extruder and the static mixer, combined, is about 0.5 to about 5 minutes and, more suitably, about 2 to about 3 minutes.

In the static mixer, the feedstock is suitable at a temperature of about 80° F. to about 150° F., a pressure of about 1200 psi to about 2500 psi and has a residence time in the static mixer of about 5 seconds to about 20 seconds.

As the fractionated feedstock passes through the die, it passes into holding vessels equipped with agitation means such as an impeller. The number of holding vessels employed in the present Invention depends on the speed at which the feedstock moves through the extruder and static mixer. Multiple holding vessels can be employed so as to make the process a continuous process. Additionally, the holding vessels can be explosive proof agitating vessels. Such vessels are conventional vessels and have agitating means such as an impeller in order to agitate the contents of the vessel. Such vessels can be air-tight so that the supercritical fluid and solvent vapors can be evacuation by vacuum from the holding tank and then recycled.

Suitably, the fractionated feedstock is held in the holding vessel at a temperature of about 80° F. to about 120° F., at a pressure of about atmospheric to below atmospheric, preferably a vacuum of about 15 bar, for a period of time of about 0.5 hours to about 1.5 hours.

The holding vessel is equipped with a heater if needed to maintain the temperature. A vacuum is created in the holding vessel in a conventional manner using conventional equipment. The vacuum helps accelerate the separation process of the solid phase and the liquid phase. The solid phase contains the starch component and the cellulosic component material whereas the liquid phase contains the protein component and the oil component of the fractionated feedstock.

After the necessary hold time for the formation of the solid phase and liquid phase, the solid phase and liquid phase are separated in a conventional manner using conventional equipment.

The solid phase which contains primarily the starch component and cellulosic component can then further be processed to strip the solvent and any remaining fluid. Suitably, a Wyssmont Type Dryer is used as shown in FIG. 2. The stripped solvent can be recycled back into the system for use again during the pressure and shearing step.

The starch and cellulosic components can then be separated into the starch and cellulose in a conventional manner using conventional equipment and then further dried as needed depending on the intended use of the solid component.

The liquid phase is further separated into the protein component and the oil component in a conventional manner using conventional equipment. Suitably, the liquids can be centrifuged and then clarified by passage through an air activated two-micron bag filter. The concentration of liquids can be formed by an explosion-proof rotovapor. The solvent which is extracted can be passed through a pre-evaporator and reclaimed to be recycled through the system. The rotovapor concentrate contains the protein component which can then be further purified in a conventional manner such as the Sessa process as shown in U.S. Pat. No. 7,939,633. The protein component can then be further dried through a vacuum drum dryer so as to provide a protein in sheets which can then be ground into a coarse particulate. The oil which stays as a liquid is recovered.

The starch that is obtained by the present Invention is a destructurized starch which means that the starch granule has been separated into individual polymeric components such as amylose and amylo pectin. Specifically, the starch as it leaves the die at the end of static mixer is destructurized.

Good results have been obtained with high amylose corn starch in the present Invention, high amylose corn starch being obtained from kernels wherein the starch component in the kernel has a high amylose content, normally 60% or more amylose while the remainder the amylose pectin.

The present Invention is found to be of competitive cost compared to the conventional wet and dry milling processes. It has also been found to reduce the stress on the grain components and to provide high quality oil and protein isolated from the plant-based product. The protein component derived from the corn kernels can be referred to as Alpha Zein.

FIG. 2 is a schematic illustration showing a twin screw extruder transitioning into the static mixer which then exits into the collection vessel or holding vessel. From the holding vessel, the fractionated feedstock is then separated into its liquid and solid phases where the liquid phase is separated into the oil.

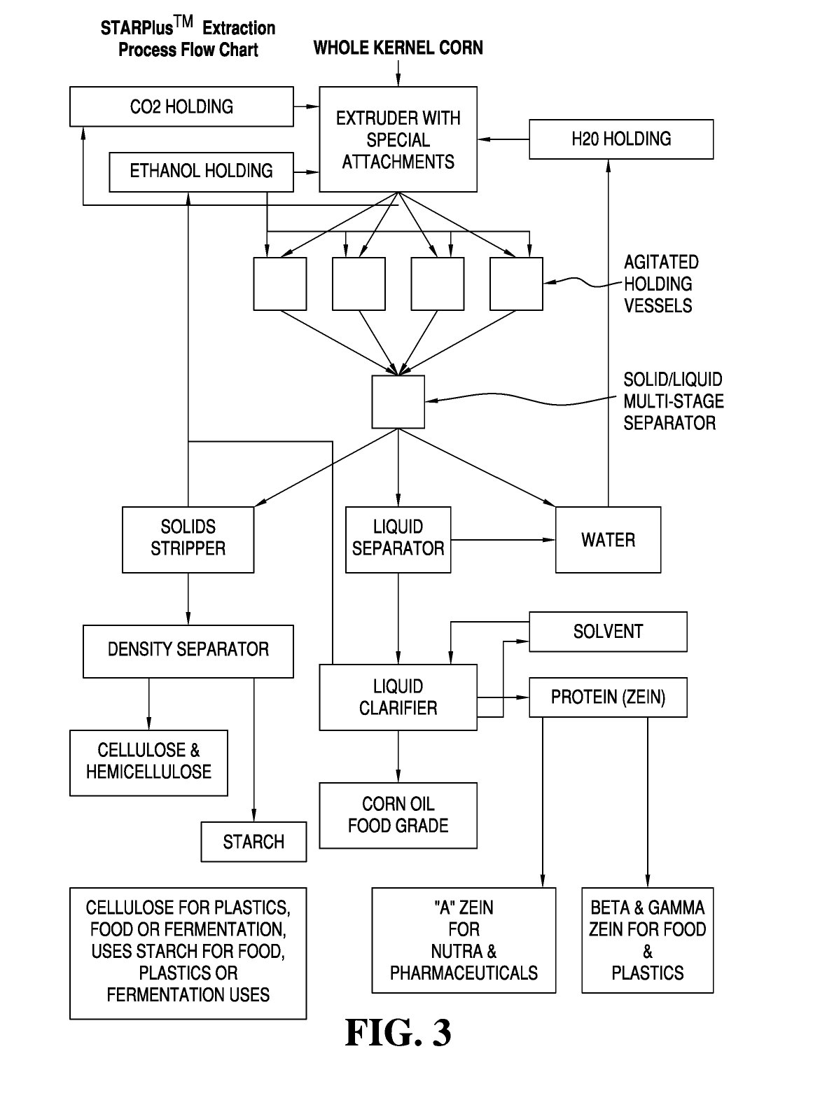

FIG. 3 illustrates a more detailed slow process of the present Invention.

I claim:

1. A method for extraction of oil and protein from a plant based product the method comprising:
forming a feedstock from a plant based product having an oil component, a protein component, a starch component and a cellulose component, and a moisture content of to about 15.5 to about 16.5% by weight;
subjecting the feedstock to pressure and shear to cause the feedstock to fractionate into an oil component, a protein component, a starch component and a cellulosic component;
holding the fractionated feedstock in a vessel with agitation for a period of time to form a solid phase having the starch component and the cellulosic component, and a liquid phase having the oil component and the protein component;
separating the solid phase from the liquid phase;
separating the liquid phase into the oil component and protein component;
collecting the oil component separate from the protein component; and
collecting the protein component separate from the oil component.

2. The method of claim 1 wherein the plant based product is a product from maize, sorghum, or wheat.

3. The method of claim 2 wherein the product is corn kernels, wheat kernels or sorghum grain.

4. The method of claim 1, wherein forming the feedstock includes determining the moisture content of the feedstock; and adjusting the moisture content of the feedstock to about 15.5% to about 16.5% by wt, if the determined moisture content is outside a range of about 15.5% to about 16.5% by wt.

5. The method of claim 1, wherein forming the feedstock includes milling the feedstock to a size of about 20 mesh to about 30 mesh.

6. The method of claim 5 wherein the milling is conducted by a hammer mill, a burr mill or a roller mill.

7. The method of claim 1, wherein subjecting the feedstock to pressure and shear includes adding a solvent to the feedstock.

8. The method of claim 1, wherein subjecting the feedstock to pressure and shear includes adding a supercritical fluid to the feedstock.

9. The method of claim 7, wherein the solvent is added in an amount of about 5 to 50% by weight based on the weight of dry feedstock.

10. The method of claim 8, wherein the supercritical fluid is added in an amount of about 10% to about 90% by weight based on the dry weight of feedstock.

11. The method of claim 1, wherein the subjecting the feedstock to pressure and shear is conducted at a temperature of about 50° F. to about 180° F., a pressure of about 1,000 psi to about 5,000 psi, and a period of time of about 0.5 minutes to about 5 minutes.

12. The method of claim 11, wherein subjecting the feedstock to pressure and shear includes feeding the feedstock to an extruder which pumps the feedstock through a static mixer.

13. The method of claim 12, wherein the extruder is a twin screw extruder operated at about 5 to 1500 rpm.

14. The method of claim 11, wherein the static mixer has an outlet with a die to restrict outflow of the fractionated feedstock.

15. The method of claim 1, wherein holding the fractioned feedstock in the vessel is conducted at a temperature of about 80° F. to about 120° F., a pressure of about atmospheric to below atmospheric, and a period of time of about 0.5 hours to about 1.5 hours.

16. The method of claim 1, wherein the separated solid phase is dried to remove the solvent.

17. The method of claim 16, wherein the starch component is separated from the cellulose component.

18. The method of claim 16, wherein the drier is a Wyssmont Type Drier.

19. The method of claim 7, wherein the solvent is absolute ethanol.

20. The method of claim 8, wherein the supercritical fluid is supercritical carbon dioxide.
